(19) United States
(12) Patent Application Publication
(10) Pub. No.: US 2025/0331827 A1
(43) Pub. Date: Oct. 30, 2025

(54) ULTRASONIC IMAGING SYSTEM AND CONTROL METHOD THEREOF

(71) Applicants: SAMSUNG MEDISON CO., LTD., Hongcheon (KR); Korea Advanced Institute of Science and Technology, Daejeon (KR)

(72) Inventors: Youngjun KO, Hongcheon (KR); Jongchul Ye, Daejeon (KR); Gunwoo Lee, Hongcheon (KR); Jaayeon Lee, Seoul (KR); Jonghyon Yi, Hongcheon (KR); Jinho Chang, Seoul (KR)

(21) Appl. No.: 19/171,327

(22) Filed: Apr. 6, 2025

(30) Foreign Application Priority Data
Apr. 30, 2024 (KR) 10-2024-0057936
Feb. 24, 2025 (KR) 10-2025-0023822

(51) Int. Cl. A61B 8/00; A61B 8/08; A61B 8/14

(52) U.S. Cl. CPC A61B 8/54; A61B 8/08; A61B 8/14; A61B 8/463; A61B 8/469; A61B 8/5207; A61B 8/5223

(57) ABSTRACT

Disclosed is an ultrasonic imaging system including a probe configured to transmit an ultrasonic signal to an object including a liver and receive an ultrasonic echo signal reflected from the object, a display configured to display an ultrasonic image, an input interface configured to obtain user input, memory configured to store an artificial intelligence model, and at least one processor. The at least one processor is configured to obtain ultrasonic raw data by processing the ultrasonic echo signal, generate a plurality of raw image frames including different characteristic information by processing the ultrasonic raw data, obtain quantitative data about a liver disease from the artificial intelligence model by inputting the plurality of raw image frames into the artificial intelligence model, and display the ultrasonic image and the quantitative data about the liver disease together on the display.

### CROSS-REFERENCE TO RELATED APPLICATION(S)

This application is based on and claims priority under 35 U.S.C. § 119 to Korean Patent Application Nos. 10-2024-0057936 and 10-2025-0023822, filed on Apr. 30, 2024 and Feb. 24, 2025, in the Korean Intellectual Property Office, the disclosure of which is incorporated by reference herein in its entirety.

### BACKGROUND

### 1. Field

The disclosure relates to an ultrasonic imaging system and a control method thereof capable of providing ultrasonic images including a variety of information using an artificial intelligence model.

### 2. Description of the Related Art

Recently, in a medical field, various medical imaging apparatuses have been widely used to image and obtain information about biological tissues of a human body for the purpose of early diagnosis of various diseases or surgery. Representative examples of such medical imaging apparatuses may include ultrasonic imaging apparatuses, computed tomography (CT) apparatuses, and magnetic resonance imaging (MRI) apparatuses.

An ultrasonic imaging apparatus is a device that emits an ultrasonic signal generated from a transducer of a probe to an object, and non-invasively obtains at least one image of a region inside the object (e.g., soft tissue or blood flow) by receiving information from the signal reflected from the object. An ultrasonic imaging apparatus may be used for medical purposes such as observing the inside of an object, detecting foreign substances, and measuring injury.

Such an ultrasonic imaging apparatus is widely used together with other imaging diagnostic apparatuses because the ultrasonic imaging apparatus has higher stability than an imaging apparatus using an X-ray, may display images in real time, and is safe because there is no radiation exposure.

Recently, various methods have emerged that utilize artificial intelligence models to process ultrasonic images.

### SUMMARY

It is an aspect of the disclosure to provide an ultrasonic imaging system and a control method thereof capable of obtaining quantitative data about a liver disease from ultrasonic raw data using an artificial intelligence model and displaying the obtained data together with an ultrasonic image.

It is an aspect of the disclosure to provide an ultrasonic imaging system and a control method thereof capable of generating a raw image including a variety of information by processing ultrasonic raw data and obtaining quantitative data about a liver disease by inputting the generated raw image into an artificial intelligence model.

Additional aspects of the disclosure will be set forth in part in the description which follows and, in part, will be obvious from the description, or may be learned by practice of the disclosure.

An aspect of the disclosure provides an ultrasonic imaging system including a probe configured to transmit an ultrasonic signal to an object including a liver and receive an ultrasonic echo signal reflected from the object; a display configured to display an ultrasonic image; an input interface configured to obtain user input; memory configured to store an artificial intelligence model; and at least one processor. The at least one processor may be configured to obtain ultrasonic raw data by processing the ultrasonic echo signal, display a first ultrasonic image generated by processing the ultrasonic raw data on the display, obtain an image frame of the first ultrasonic image in response to obtaining a freezing command through the input interface, generate a plurality of raw image frames corresponding to the obtained image frame and including different characteristic information by processing the ultrasonic raw data, obtain quantitative data about a liver disease from the artificial intelligence model by inputting the plurality of raw image frames into the artificial intelligence model, and display a second ultrasonic image including the quantitative data about the liver disease on the display.

The at least one processor may be configured to generate a B-mode image as the first ultrasonic image, and generate a first raw image frame including attenuation information of the ultrasonic echo signal and a second raw image frame including scattering information of the ultrasonic echo signal, as the plurality of raw image frames.

The at least one processor may be further configured to generate at least one of a third raw image frame including in-phase component information and quadrature component information of the ultrasonic raw data and a fourth raw image frame including frequency spectrum information of the ultrasonic raw data, as the plurality of raw image frames.

The at least one processor may be configured to identify a first region of interest in the image frame of the first ultrasonic image, determine a second region of interest corresponding to the first region of interest in each of the plurality of raw image frames, extract a partial image frame corresponding to the second region of interest in each of the plurality of raw image frames, and obtain the quantitative data about the liver disease in the first region of interest by inputting a plurality of the partial image frames corresponding to each of a plurality of the second regions of interest into the artificial intelligence model.

The at least one processor may be configured to obtain first coordinate information of the first region of interest in the image frame of the first ultrasonic image, convert the first coordinate information into second coordinate information in each of the plurality of raw image frames, and determine the second region of interest based on the second coordinate information.

The at least one processor may be configured to obtain at least one of a fat fraction of the liver and severity of liver steatosis as the quantitative data about the liver disease.

The at least one processor may be configured to obtain additional information about the object including at least one of a subcutaneous fat thickness, a body mass index (BMI), gender, age, and an underlying disease through the input interface, and input the plurality of raw image frames and the additional information about the object into the artificial intelligence model.

The at least one processor may be configured to display the quantitative data about the liver disease in at least one of a first region of the display displaying the first ultrasonic image and the second ultrasonic image and a second region of the display divided from the first region.

The at least one processor may be further configured to display a heat map visualizing a distribution of the quantitative data about the liver disease in the first ultrasonic image on the display.

The at least one processor may be configured to display the second ultrasonic image on the display based on obtaining a command for activating an artificial intelligence-based function through the input interface.

Another aspect of the disclosure provides a control method of an ultrasonic imaging system, which includes a probe, an input interface, a display, and at least one processor, including a control method executed by the at least one processor, wherein the control method may include controlling the probe to transmit an ultrasonic signal to an object including a liver and receive an ultrasonic echo signal reflected from the object; obtaining ultrasonic raw data by processing the ultrasonic echo signal; displaying a first ultrasonic image generated by processing the ultrasonic raw data on the display; obtaining an image frame of the first ultrasonic image in response to obtaining a freezing command through the input interface; generating a plurality of raw image frames corresponding to the obtained image frame and including different characteristic information by processing the ultrasonic raw data; obtaining quantitative data about a liver disease from the artificial intelligence model by inputting the plurality of raw image frames into the artificial intelligence model; and displaying a second ultrasonic image including the quantitative data about the liver disease on the display.

The first ultrasonic image may correspond to a B-mode image, and the generating of the plurality of raw image frames may include generating a first raw image frame including attenuation information of the ultrasonic echo signal and a second raw image frame including scattering information of the ultrasonic echo signal.

The generating of the plurality of raw image frames may further include generating at least one of a third raw image frame including in-phase component information and quadrature component information of the ultrasonic raw data and a fourth raw image frame including frequency spectrum information of the ultrasonic raw data, as the plurality of raw image frames.

The obtaining of the quantitative data about the liver disease may include identifying a first region of interest in the image frame of the first ultrasonic image; determining a second region of interest corresponding to the first region of interest in each of the plurality of raw image frames; extracting a partial image frame corresponding to the second region of interest in each of the plurality of raw image frames; and obtaining the quantitative data about the liver disease in the first region of interest by inputting a plurality of the partial image frames corresponding to each of a plurality of the second regions of interest into the artificial intelligence model.

The determining of the second region of interest may include obtaining first coordinate information of the first region of interest in the image frame of the first ultrasonic image; converting the first coordinate information into second coordinate information in each of the plurality of raw image frames; and determining the second region of interest based on the second coordinate information.

The quantitative data about the liver disease may include at least one of a fat fraction of the liver and severity of liver steatosis.

The control method may further include obtaining additional information about the object including at least one of a subcutaneous fat thickness, a body mass index (BMI), gender, age, and an underlying disease through the input interface; and inputting the plurality of raw image frames and the additional information about the object into the artificial intelligence model.

The displaying of the second ultrasonic image may include displaying the quantitative data about the liver disease in at least one of a first region of the display displaying the first ultrasonic image and the second ultrasonic image and a second region of the display divided from the first region.

The control method may further include displaying a heat map visualizing a distribution of the quantitative data about the liver disease in the first ultrasonic image on the display.

The displaying of the second ultrasonic image may be performed based on obtaining a command for activating an artificial intelligence-based function through the input interface.

### BRIEF DESCRIPTION OF THE DRAWINGS

These and/or other aspects of the disclosure will become apparent and more readily appreciated from the following description of the embodiments, taken in conjunction with the accompanying drawings of which:

FIGS. 1A and 1B are block diagrams illustrating components of an ultrasonic imaging system according to various embodiments;

FIGS. 2A, 2B, 2C, and 2D illustrate an ultrasonic imaging apparatus according to various embodiments;

FIG. 3 is a flowchart for explaining a control method of the ultrasonic imaging system according to an embodiment;

FIG. 4 is a diagram for explaining a process in which an artificial intelligence-based function of the ultrasonic imaging system according to an embodiment is performed;

FIG. 5 illustrates an example of an aspect in which an ultrasonic image and quantitative data about a liver disease are displayed through a display of the ultrasonic imaging apparatus;

FIG. 6 illustrates an example of an aspect in which an ultrasonic image and quantitative data about a liver disease are displayed through the display of the ultrasonic imaging apparatus;

FIG. 7 illustrates an example of an aspect in which an ultrasonic image and quantitative data about a liver disease are displayed through the display of the ultrasonic imaging apparatus; and

FIG. 8 illustrates an example of an aspect in which an ultrasonic image and quantitative data about a liver disease are displayed through the display of the ultrasonic imaging apparatus.

### DETAILED DESCRIPTION

This disclosure will explain embodiments of the disclosure to clarify the scope of the claims of the disclosure and enable those skilled in the art to which the embodiments of the disclosure belong to practice the embodiments.

Throughout the specification, like reference numbers refer to like elements throughout this specification. This specification does not describe all components of the embodiments, and general contents in the technical field to which the disclosure belongs or overlapping contents between the embodiments will not be described. The “module” or “unit” used in the specification may be implemented as one or a combination of two or more of software, hardware, or firmware, and according to embodiments, a plurality of “module” or “unit” may be implemented as a single element, or a single “module” or “unit” may include a plurality of elements.

The singular form of a noun corresponding to an item may include a single item or a plurality of items, unless the relevant context clearly indicates otherwise.

In this disclosure, each of phrases such as “A or B,” “at least one of A and B,” “at least one of A or B,” “A, B or C,” “at least one of A, B and C,” and “at least one of A, B, or C” may include any one of the items listed together in the corresponding one of the phrases, or all possible combinations thereof.

The term “and/or” includes any combination of a plurality of related components or any one of a plurality of related components.

For example, expressions such as “A and/or B” or “at least one of A or B” may include all possible combinations of items listed together. For example, “A and/or B” or “at least one of A or B” may refer to all cases in which (1) only A is included, (2) only B is included, or (3) both A and B are included.

The terms such as “first,” “second,” “primary,” and “secondary” may simply be used to distinguish a given component from other corresponding components, and do not limit the corresponding components in any other respect (e.g., importance or order).

The terms “front surface,” “rear surface,” “upper surface,” “lower surface,” “side surface,” “left side,” “right side,” “upper portion,” “lower portion,” and the like used in the disclosure are defined with reference to the drawings, and the shape and position of each component are not limited by these terms.

The terms “comprises,” “has,” and the like are intended to indicate that there are features, numbers, steps, operations, components, parts, or combinations thereof described in the disclosure, and do not exclude the presence or addition of one or more other features, numbers, steps, operations, components, parts, or combinations thereof.

When any component is referred to as being “connected,” “coupled,” “supported,” or “in contact” with another component, this includes a case in which the components are indirectly connected, coupled, supported, or in contact with each other through a third component as well as directly connected, coupled, supported, or in contact with each other.

When any component is referred to as being located “on” or “over” another component, this includes not only a case in which any component is in contact with another component but also a case in which another component is present between the two components.

In this disclosure, an ‘object’, which is subject to photography, may include a person, animal, or part thereof. For example, the object may include a part of a human body (an organ, etc.) or a phantom.

In this disclosure, an ‘ultrasonic image’ refers to an image of an object that has been generated or processed based on an ultrasonic signal (echo signal) transmitted to and reflected from the object.

In this disclosure, ‘visual indicators’ may include various indicators such as letters, numbers, shapes (points, lines, surfaces, three-dimensional structures), colors, animations, and visual effects.

Hereinafter, embodiments will be described in detail with reference to the drawings.

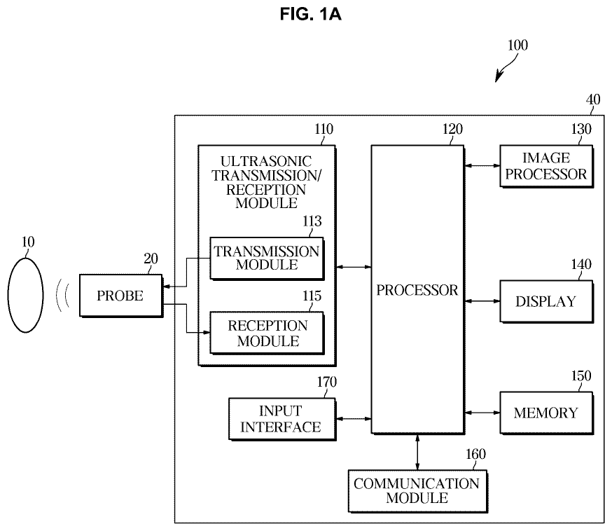

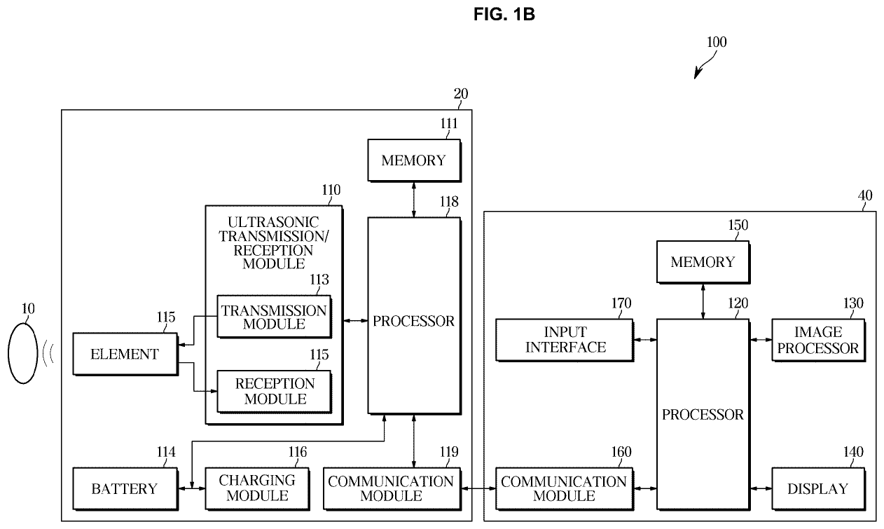

FIGS. 1A and 1B are block diagrams illustrating components of an ultrasonic imaging system according to various embodiments.

Referring to FIGS. 1A and 1B, an ultrasonic imaging system 100 may include a probe 20 and an ultrasonic imaging apparatus 40.

The ultrasonic imaging apparatus 40 may be implemented not only in a cart type but also in a portable type. A portable ultrasonic imaging apparatus may include, for example, a smart phone, a laptop computer, a personal digital assistant (PDA), a tablet PC, etc., which include a probe and an application, but is not limited thereto. The ultrasonic imaging apparatus 40 may also be implemented as an integrated probe.

The probe 20 may include a wired probe connected to the ultrasonic imaging apparatus 40 by wire to communicate with the ultrasonic imaging apparatus 40 by wire, a wireless probe wirelessly connected to the ultrasonic imaging apparatus 40 to communicate wirelessly with the ultrasonic imaging apparatus 40, and/or a hybrid probe connected to the ultrasonic imaging apparatus 40 by wire or wirelessly to communicate with the ultrasonic imaging apparatus 40 by wire or wirelessly. The probe 20 may be referred to as an ‘ultrasonic probe’ in that it transmits and receives ultrasonic signals.

According to various embodiments, as illustrated in FIG. 1A, the ultrasonic imaging apparatus 40 may include an ultrasonic transmission/reception module 110. As illustrated in FIG. 1B, the probe 20 may also include the ultrasonic transmission/reception module 110. According to various embodiments, both the ultrasonic imaging apparatus 40 and the probe 20 may also include the ultrasonic transmission/reception module 110.

According to various embodiments, the probe 20 may further include at least one or a combination of an image processor 130, a display 140, and an input interface 170. Descriptions of the ultrasonic transmission/reception module 110, the image processor 130, the display 140, and the input interface 170 included in the ultrasonic imaging apparatus 40 may also be applied to the ultrasonic transmission/reception module 110, the image processor 130, the display 140, and the input interface 170 included in the probe 20.

FIG. 1A illustrates a block diagram illustrating components of the ultrasonic imaging system 100 in a case in which the probe 20 is a wired probe or a hybrid probe. In the case in which the probe 20 is a wired probe or a hybrid probe, the probe 20 may include a cable and connector connectable to a connector of the ultrasonic imaging apparatus 40.

The probe 20 may include a plurality of transducers. The plurality of transducers may be arranged in a predetermined arrangement to be implemented as a transducer array. The transducer array may correspond to a one-dimensional (1D) array or a two-dimensional (2D) array. The plurality of transducers may transmit an ultrasonic signal to an object 10 in response to a transmission signal applied from a transmission module 113. The plurality of transducers may form a reception signal by receiving the ultrasonic signal (echo signal) reflected from the object 10. The probe 20 may be implemented as an integrated type with the ultrasonic imaging apparatus 40, or may be implemented as a separate type connected to the ultrasonic imaging apparatus 40 by wire. The ultrasonic imaging apparatus 40 may be connected to the one or more probes 20 depending on the implementation type.

The probe 20 may be implemented as a two-dimensional probe. In a case in which the probe 20 is implemented as a two-dimensional probe, the plurality of transducers included in the probe 20 may be arranged in two dimensions to form a two-dimensional transducer array. For example, the two-dimensional transducer array may have a form in which a plurality of sub-arrays including the plurality of transducers arranged in a first direction is arranged in a second direction different from the first direction.

In the case in which the probe 20 is implemented as a two-dimensional probe, the ultrasonic transmission/reception module 110 may include at least one of an analog beamformer and a digital beamformer. Also, the two-dimensional probe may include at least one or a combination of the analog beamformer and the digital beamformer depending on the implementation type.

A processor 120 controls the transmission module 113 to form a transmission signal to be applied to each of transducers 117 in consideration of positions and focused points of the plurality of transducers included in the probe 20.

The processor 120 may control a reception module 115 to generate ultrasonic data by converting reception signals received from the probe 20 into analog to digital and summing up the digitally converted reception signals in consideration of the positions and focused points of the plurality of transducers. The ultrasonic data may include ultrasonic raw data and/or ultrasonic image data generated based on the ultrasonic raw data. The ultrasonic raw data may also be referred to as RF data.

In the case in which the probe 20 is implemented as a two-dimensional probe the processor 120 may calculate a time delay value for digital beamforming for each sub-array for each of the plurality of sub-arrays included in the two-dimensional transducer array. The processor 120 may also calculate a time delay value for analog beamforming for each of the transducers included in one of the plurality of sub-arrays. The processor 120 may control the analog beamformer and the digital beamformer to form a transmission signal to be applied to each of the plurality of transducers depending on the time delay values for analog beamforming and the time delay values for digital beamforming. The processor 120 may also control the analog beamformer to sum up the signals received from the plurality of transducers for each sub-array depending on the time delay values for analog beamforming. The processor 120 may also control the ultrasonic transmission/reception module 110 to convert the summed signal for each sub-array into analog to digital. The processor 120 may also control the digital beamformer to generate ultrasonic data by summing up the digitally converted signals depending on the time delay values for digital beamforming.

The image processor 130 may generate and/or process an ultrasonic image using the generated ultrasonic data. The processing of the ultrasonic image by the image processor 130 may include processing ultrasonic raw data by the image processor 130 to generate a raw image frame including a variety of information and/or generate ultrasonic images in various modes. The ultrasonic raw data may also be referred to as RF data.

Ultrasonic images may be provided in various modes. For example, modes for ultrasonic images may include an amplitude mode (A-mode), a brightness mode (B-mode), a color Doppler mode, a Doppler mode (D-mode), an elastography mode (E-mode), a motion mode (M-mode), and a volume mode. Depending on a mode for an ultrasonic image, a method of processing ultrasonic raw data may vary.

The display 140 may display the generated ultrasonic image and a variety of information processed in the ultrasonic imaging apparatus 40 or the probe 20. The probe 20 or the ultrasonic imaging apparatus 40 may include the one or more displays 140 depending on the implementation type. The display 140 may also include a touch panel or a touch screen. The display 140 may also include a flexible display.

It is also possible for the probe 20 itself to have the display 140.

The processor 120 may control the overall operation of the ultrasonic imaging apparatus 40 and control operations of components of the ultrasonic imaging apparatus 40. The processor 120 may perform or control various operations and/or functions of the ultrasonic imaging apparatus 40 by executing programs or instructions stored in memory 150. The processor 120 may also control an operation of the ultrasonic imaging apparatus 40 by receiving a control signal from the input interface 170 or an external device.

The ultrasonic imaging apparatus 40 may include a communication module 160, and may be connected to and communicate with an external device (e.g., a probe, a server, a computing apparatus, a medical device, a portable device (a smart phone, tablet PC, wearable device, etc.) through the communication module 160.

The communication module 160 may include one or more components enabling communication with the external device. The communication module 160 may include, for example, at least one of a short-range communication module, a wired communication module, and a wireless communication module.

The communication module 160 may receive a control signal or data from the external device. The processor 120 may control the operation of the ultrasonic imaging apparatus 40 according to a control signal received through the communication module 160. Also, the processor 120 may transmit a control signal to the external device through the communication module 160, thereby controlling the external device according to the transmitted control signal. The external device may operate according to the control signal received from the ultrasonic imaging apparatus 40 or may process data received from the ultrasonic imaging apparatus 40.

A program or application related to the ultrasonic imaging apparatus 40 may be installed in the external device. The program or application installed in the external device may control the ultrasonic imaging apparatus 40 or operate according to the control signal or data received from the ultrasonic imaging apparatus 40.

The external device may receive or download the program or application related to the ultrasonic imaging apparatus 40 from the ultrasonic imaging apparatus 40, the probe 20, or a computing apparatus 30 to install and execute the program or application in the external device. The ultrasonic imaging apparatus 40 or the probe 20 providing the program or application may include a recording medium storing instructions, commands, installation files, executable files, or related data of the corresponding program or application. The external device may also be sold with the program or application installed.

The memory 150 may store various data or programs for driving and controlling the ultrasonic imaging apparatus 40, inputted and outputted ultrasonic data, ultrasonic images, and the like.

The input interface 170 may receive user input for controlling the ultrasonic imaging apparatus 40. For example, the user input may include, but is not limited to, input of manipulating a button, a keypad, a dial, a mouse, a trackball, a jog switch, a knob, and the like, input of touching a touch pad or touch screen, voice input, motion input, biometric information input (e.g., iris recognition, fingerprint recognition, etc.), and the like.

FIG. 1B illustrates a control block diagram of the ultrasonic imaging system 100 in a case in which the probe 20 is a wireless probe or a hybrid probe. According to various embodiments, the ultrasonic imaging apparatus 40 illustrated in FIG. 1B may be replaced with the ultrasonic imaging apparatus 40 described with reference to FIG. 1A. The probe 20 described with reference to FIG. 1A may be replaced with the probe 20 to be described with reference to FIG. 1B.

The probe 20 may include a display 112, the transmission module 113, a battery 114, the transducer 117, a charging module 116, the reception module 115, an input interface 109, a processor 118, and a communication module 119. FIG. 1B illustrates, but is not limited to, the probe 20 including both the transmission module 113 and the reception module 115. The probe 20 may include only part of a configuration of the transmission module 113 and the reception module 115. The part of the configuration of the transmission module 113 and the reception module 115 may be included in the ultrasonic imaging apparatus 40. Additionally, the probe 20 may further include the image processor 130.

The transducer 117 may include a plurality of transducers. The plurality of transducers may be arranged in a predetermined arrangement to be implemented as a transducer array. The transducer array may correspond to a one-dimensional (1D) array or a two-dimensional (2D) array. The plurality of transducers may transmit ultrasonic signals to the object 10 in response to transmission signals applied from the transmission module 113. The plurality of transducers may also receive ultrasonic signals reflected from the object 10 to form or generate electrical reception signals.

The charging module 116 may charge the battery 114. The charging module 116 may receive electric power from the outside. The charging module 116 may receive electric power wirelessly. The charging module 116 may also receive electric power by wire. The charging module 116 may transfer the received electric power to the battery 114.

The processor 118 controls the transmission module 113 to generate or form a transmission signal to be applied to each of the plurality of transducers in consideration of the positions and focused points of the plurality of transducers.

The processor 118 controls the reception module 115 to generate ultrasonic data by converting reception signals received from the transducer 117 into analog to digital and summing up the digitally converted reception signals in consideration of the positions and focused points of the plurality of transducers. According to an embodiment of the disclosure, in a case in which the probe 20 includes the image processor 130, the probe 20 may generate an ultrasonic image using the generated ultrasonic data.

In the case in which the probe 20 is implemented as a two-dimensional probe, the processor 118 may calculate the time delay value for digital beamforming for each sub-array for each of the plurality of sub-arrays included in the two-dimensional transducer array. The processor 118 may also calculate the time delay value for analog beamforming for each of the transducers included in one of the plurality of sub-arrays. The processor 118 may control the analog beamformer and the digital beamformer to form a transmission signal to be applied to each of the plurality of transducers depending on the time delay values for analog beamforming and the time delay values for digital beamforming. The processor 118 may also control the analog beamformer to sum up the signals received from the plurality of transducers for each sub-array depending on the time delay values for analog beamforming. The processor 118 may also control the ultrasonic transmission/reception module 110 to convert the summed signal for each sub-array into analog to digital. The processor 118 may also control the digital beamformer to generate ultrasonic data by summing up the digitally converted signals depending on the time delay values for digital beamforming.

The processor 118 may control the overall operation of the probe 20 and control operations of the components of the probe 20. The processor 118 may perform or control various operations or functions of the probe 20 by executing programs or instructions stored in memory 111. The processor 118 may also control an operation of the probe 20 by receiving a control signal from the input interface 109 of the probe 20 or an external device (e.g., the ultrasonic imaging apparatus 40). The processor 118 may also control the operation of the probe 20 by receiving a control signal from the input interface 109 or the external device. The input interface 109 may receive user input for controlling the probe 20. For example, the user input may include, but is not limited to, input of manipulating a button, a keypad, a mouse, a trackball, a jog switch, a knob, and the like, input of touching a touch pad or touch screen, voice input, motion input, biometric information input (e.g., iris recognition, fingerprint recognition, etc.), and the like.

The display 112 may display an ultrasonic image generated by the probe 20, an ultrasonic image generated by processing ultrasonic data generated in the probe 20, an ultrasonic image received from the ultrasonic imaging apparatus 40, or a variety of information processed in the ultrasonic imaging system 100. The display 112 may also display state information of the probe 20. The state information of the probe 20 may include at least one of device information of the probe 20, battery state information of the probe 20, frequency band information of the probe 20, output information of the probe 20, information on whether the probe 20 is abnormal, setting information of the probe 20, and temperature information of the probe 20.

The probe 20 may include the one or more displays 112 depending on the implementation type. The display 112 may include a touch panel or a touch screen. The display 112 may also include a flexible display.

The communication module 119 may wirelessly transmit the generated ultrasonic data or ultrasonic images to the ultrasonic imaging apparatus 40 through a wireless network. The communication module 119 may also receive a control signal and data from the ultrasonic imaging apparatus 40.

The ultrasonic imaging apparatus 40 may receive the ultrasonic data and/or ultrasonic images from the probe 20.

In the case in which the probe 20 includes the image processor 130 capable of generating ultrasonic images using the ultrasonic data, the probe 20 may transmit the ultrasonic data or the ultrasonic images generated by the image processor 130 to the ultrasonic imaging apparatus 40.

In a case in which the probe 20 does not include the image processor 130 capable of generating ultrasonic images using the ultrasonic data, the probe 20 may transmit the ultrasonic data to the ultrasonic imaging apparatus 40.

The ultrasonic imaging apparatus 40 may include the processor 120, the image processor 130, the display 140, the memory 150, the communication module 160, and the input interface 170.

The image processor 130 generates and/or processes ultrasonic images using the ultrasonic data received from the probe 20.

The display 140 may display the ultrasonic images received from the probe 20, ultrasonic images generated by processing the ultrasonic data received from the probe 20, and/or a variety of information processed by the ultrasonic imaging system 100. The ultrasonic imaging apparatus 40 may include the one or more displays 140 depending on the implementation type. The display 140 may also include a touch panel or a touch screen. The display 140 may also include a flexible display.

The processor 120 may control the overall operation of the ultrasonic imaging apparatus 40 and control the operations of the components of the ultrasonic imaging apparatus 40. The processor 120 may perform or control the various operations or functions of the ultrasonic imaging apparatus 40 by executing the programs or applications stored in the memory 150. The processor 120 may also control the operation of the ultrasonic imaging apparatus 40 by receiving a control signal from the input interface 170 or an external device.

The ultrasonic imaging apparatus 40 may include the communication module 160, and may be connected to and communicate with an external device (e.g., the probe 20, the computing apparatus, a medical device, a portable device (a smart phone, tablet PC, wearable device, etc.)) through the communication module 160.

The communication module 160 may include one or more components that enable communication with the external device. The communication module 160 may include, for example, at least one of the short-range communication module, the wired communication module, and the wireless communication module.

The communication module 160 of the ultrasonic imaging apparatus 40 and the communication module 119 of the probe 20 may communicate using a network or a short-range wireless communication method. For example, the communication module 160 of the ultrasonic imaging apparatus 40 and the communication module 119 of the probe 20 may communicate using any one of wireless LAN, Wi-Fi, Bluetooth, ZigBee, Wi-Fi Direct (WFD), Infrared Data Association (IrDA), Bluetooth Low Energy (BLE), Near Field Communication (NFC), Wireless Broadband Internet (WiBro), World Interoperability for Microwave Access (WiMAX), Shared Wireless Access Protocol (SWAP), Wireless Gigabit Alliance (WiGig), RF communication, a wireless data communication method including 60 GHz millimeter wave (mm wave) short-range communication, etc.

To this end, the communication module 160 of the ultrasonic imaging apparatus 40 and the communication module 119 of the probe 20 may include at least one of a wireless LAN communication module, a Wi-Fi communication module, a Bluetooth communication module, a ZigBee communication module, a Wi-Fi Direct (WFD) communication module, an Infrared Data Association (IrDA) communication module, a Bluetooth Low Energy (BLE) communication module, a Near Field Communication Broadband Internet (WiBro) communication module, a World Interoperability for Microwave Access (WiMAX) communication module, a Shared Wireless Access Protocol (SWAP) communication module, a Wireless Gigabit Alliance (WiGig) communication module, a RF communication module, and a 60 GHz millimeter wave (mm wave) short-range communication module.

The probe 20 may transmit device information (e.g., ID information) of the probe 20 to the ultrasonic imaging apparatus 40 using a first communication method (e.g., BLE) and may be wirelessly paired with the ultrasonic imaging apparatus 40. The probe 20 may also transmit ultrasonic data and/or ultrasonic images to the paired ultrasonic imaging apparatus 40. The device information of the probe 20 may include a variety of information about a serial number, model name, or battery state of the probe 20. The probe 20 may also transmit the ultrasonic data and/or ultrasonic images to the paired ultrasonic imaging apparatus 40 by the first communication method using a second communication method (e.g., 60 GHz millimeter wave and Wi-Fi).

The ultrasonic imaging apparatus 40 may receive the device information (e.g., ID information) of the probe 20 from the probe 20 using the first communication method (e.g., BLE) and may be wirelessly paired with the probe 20. The ultrasonic imaging apparatus 40 may also transmit an activation signal to the paired probe 20 and receive the ultrasonic data and/or ultrasonic images from the probe 20. In this case, the activation signal may include a signal for controlling the operation of the probe 20. The ultrasonic imaging apparatus 40 may also transmit the activation signal to the paired probe 20 and receive the ultrasonic data and/or ultrasonic images from the probe 20 using the second communication method (e.g., 60 GHz millimeter wave and Wi-Fi).

The first communication method used to pair the probe 20 and the ultrasonic imaging apparatus 40 with each other may have a frequency band lower than a frequency band of the second communication method used by the probe 20 to transmit the ultrasonic data and/or ultrasonic images to the ultrasonic imaging apparatus 40.

The display 140 of the ultrasonic imaging apparatus 40 may display UIs (user interfaces) indicating the device information of the probe 20. For example, the display 140 may display UIs, which indicate identification information of the wireless probe 20, a pairing method indicating a pairing method with the probe 20, a data communication state between the probe 20 and the ultrasonic imaging apparatus 40, a method of performing data communication with the ultrasonic imaging apparatus 40, and/or UI indicating the battery state of the probe 20.

In a case in which the probe 20 includes the display 112, the display 112 of the probe 20 may display the UIs indicating the device information of the probe 20. For example, the display 112 may display UIs, which indicate the identification information of the wireless probe 20, the pairing method indicating the pairing method with the probe 20, the data communication state between the probe 20 and the ultrasonic imaging apparatus 40, the method of performing the data communication with the ultrasonic imaging apparatus 40, and/or the UI indicating the battery state of the probe 20.

The communication module 160 may receive a control signal or data from an external device. The processor 120 may control the operation of the ultrasonic imaging apparatus 40 in response to the control signal received through the communication module 160.

Also, the processor 120 may transmit a control signal to the external device through the communication module 160 to control the external device according to the transmitted control signal. The external device may operate according to a control signal received from the ultrasonic imaging apparatus 40 or process data received from the ultrasonic imaging apparatus 40.

The external device may receive or download the program or application related to the ultrasonic imaging apparatus 40 from the ultrasonic imaging apparatus 40 or the probe 20 to install and execute the program or application in the external device. The ultrasonic imaging apparatus 40 or the probe 20 providing the program or application may include a recording medium storing instructions, commands, installation files, executable files, or related data of the program or application. The external device may be sold with the program or application installed.

The memory 150 may store various data or programs for driving and controlling the ultrasonic imaging apparatus 40, inputted and outputted ultrasonic data, ultrasonic images, etc.

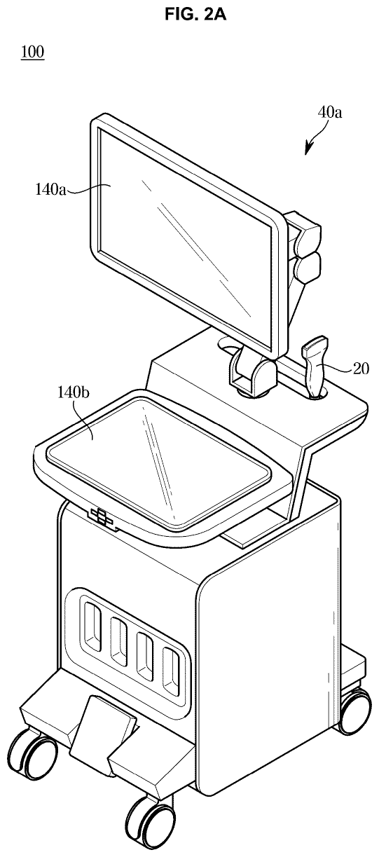

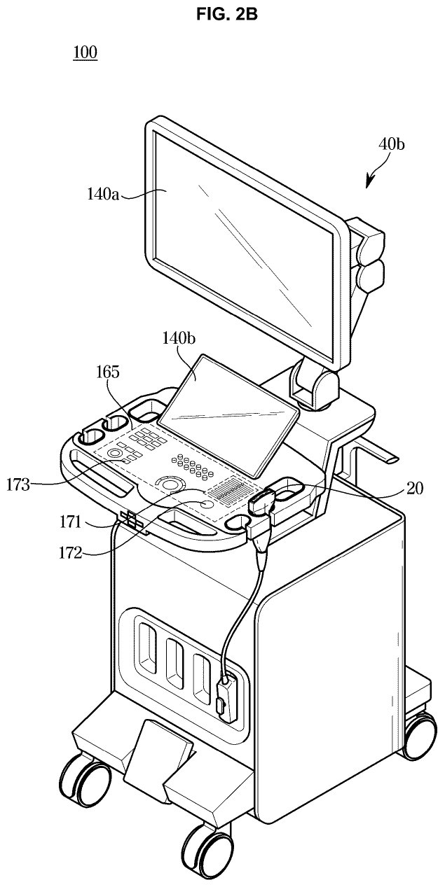

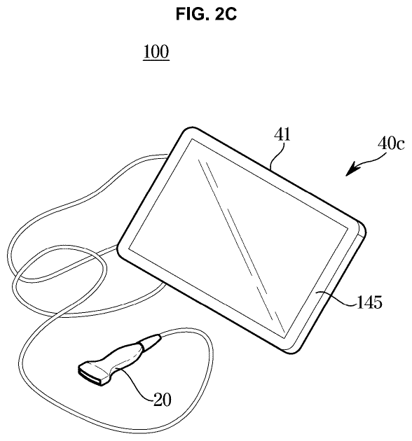

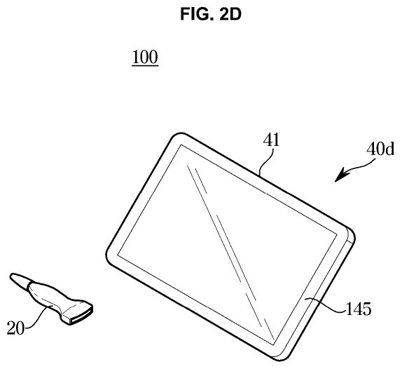

FIGS. 2A, 2B, 2C, and 2D illustrate an ultrasonic imaging apparatus according to various embodiments.

Referring to FIGS. 2A and 2B, ultrasonic imaging apparatuses 40a and 40b may include a main display 140a and a sub display 140b. The main display 140a and the sub display 140b may correspond to the display 140 of FIGS. 1A and 1B. At least one of the main display 140a and the sub display 140b may be implemented as a touch screen. At least one of the main display 140a and the sub display 140b may display ultrasonic images or a variety of information processed in the ultrasonic imaging apparatuses 40a and 40b.

At least one of the main display 140a and the sub display 140b may be implemented as a touch screen and provide GUIs (graphic user interfaces), so that data for controlling the ultrasonic imaging apparatuses 40a and 40b may be inputted from a user. For example, the main display 140a may display ultrasonic images, and the sub display 140b may display a control panel for controlling the display of the ultrasonic images in the form of GUIs. Data for controlling the display of images may be inputted into the sub display 140b through the control panel displayed in the form of GUIs.

For example, the GUIs provided through the sub display 140b may include a time gain compensation (TGC) button, a lateral gain compensation (LGC) button, a freeze button, a trackball, a jog switch, a knob and/or an artificial intelligence-based function button 173.

The ultrasonic imaging apparatuses 40a and 40b may control the display of ultrasonic images displayed on the main display 140a using the inputted control data. The ultrasonic imaging apparatuses 40a and 40b may also be connected to the probe 20 by wire or wirelessly to transmit and receive ultrasonic signals to and from the object.

Referring to FIG. 2B, the ultrasonic imaging apparatus 40b may further include a control panel 165 in addition to the main display 140a and the sub display 140b. The control panel 165 may include a button, a trackball, a jog switch, a knob, and the like, and data for controlling the ultrasonic imaging apparatus 40b may be inputted into the control panel 165 from the user.

For example, the control panel 165 may include a TGC button 171, a freeze button 172, and/or the artificial intelligence-based function button 173. The TGC button 171 is a button for setting a TGC value for each depth of ultrasonic images.

Additionally, the ultrasonic image apparatus 40b may obtain a freezing command through the freeze button 172 while displaying an ultrasonic image including a plurality of image frames. The ultrasonic image apparatus 40b may pause the display of the ultrasonic image and display an image frame at the point in time when the freezing command is obtained. The ultrasonic imaging apparatus 40b may capture the image frame at the point in time when the freezing command is obtained. The ultrasonic imaging apparatus 40b may also store the image frame at the point in time when the freezing command is obtained.

The artificial intelligence-based function button 173 may be a button for activating one of various artificial intelligence-based functions. The artificial intelligence-based function may refer to a function of using an artificial intelligence model. For example, the artificial intelligence-based function may include functions such as a disease diagnosis function, an image segmentation function, an image improvement function, a fetal analysis function, a body marker setting function, a parameter measurement function, and/or a function of obtaining quantitative data about a liver disease.

Various input devices such as a button, a trackball, a jog switch, a knob included in the control panel 165 may be provided as GUIs on the main display 140a and/or the sub display 140b. The ultrasonic imaging apparatuses 40a and 40b may be connected to the probe 20 to transmit and receive ultrasonic signals to and from the object.

The ultrasonic imaging apparatus 40a and 40b may include various types of output interfaces such as speakers, LEDs, and vibration devices. For example, the ultrasonic imaging apparatus 40a and 40b may output a variety of information in the form of graphics, sound, or vibration through the output interfaces. The ultrasonic imaging apparatus 40a and 40b may also output various notifications or data through the output interfaces.

Referring to FIGS. 2C and 2D, ultrasonic imaging apparatuses 40c and 40d may be implemented in a portable type. The portable ultrasonic imaging apparatuses 40c and 40d may include, for example, smart phones, laptop computers, PDAs, or tablet PCs, which include probes and applications, but is not limited thereto.

The ultrasonic imaging apparatus 40c may include a main body 41. Referring to FIG. 20, the probe 20 may be connected to one side of the main body 41 by wire. To this end, the main body 41 may include a connection terminal to and from which a cable connected to the probe 20 may be attached and detached. The probe 20 may include a cable including a connection terminal capable of being connected to the main body 41.

Referring to FIG. 2D, the probe 20 may be wirelessly connected to the ultrasonic imaging apparatus 40d. The main body 41 may include an input/output interface (e.g., a touch screen) 145. Ultrasonic images, a variety of information, and/or GUIs may be displayed on the input/output interface 145.

The ultrasonic imaging apparatus 40d and the probe 20 may establish communication or be paired using a short-range wireless communication. For example, the ultrasonic imaging apparatus 40d and the probe 20 may perform communication using Bluetooth, BLE, Wi-Fi, or Wi-Fi Direct.

The ultrasonic imaging apparatuses 40c and 40d may execute a program or application related to the probe 20 to control the probe 20 and output information about the probe 20. The ultrasonic imaging apparatuses 40c and 40d may perform operations related to the probe 20 while communicating with the predetermined computing apparatus 30. The probe 20 may be registered with the ultrasonic imaging apparatuses 40c and 40d or may be registered with the predetermined computing apparatus 30. The ultrasonic imaging apparatuses 40c and 40d may communicate with the registered probe 20 and perform the operations related to the probe 20.

The ultrasonic imaging apparatuses 40c and 40d may also include various types of output interfaces such as speakers, LEDs, and vibration devices. For example, the ultrasonic imaging apparatuses 40c and 40d may output a variety of information in the form of graphics, sound, or vibration through the output interfaces. The ultrasonic imaging apparatuses 40c and 40d may also output various notifications or data through the output interfaces.

The ultrasonic imaging apparatuses 40a, 40b, 40c, and 40d according to various embodiments may process an ultrasonic image using an artificial intelligence (AI) model. The ultrasonic imaging apparatuses 40a, 40b, 40c, and 40d may also obtain a variety of information from ultrasonic images. The AI model may be stored in the ultrasonic imaging apparatuses 40a, 40b, 40c, and 40d.

For example, the ultrasonic imaging apparatuses 40a, 40b, 40c, and 40d may, using the AI model, perform image processing such as generating ultrasonic images, correcting ultrasonic images, improving image quality, encoding, and/or decoding. The ultrasonic imaging apparatuses 40a, 40b, 40c, and 40d may also, using the AI model, perform quantitative data obtainment on diseases of various tissues, reference line definition, anatomical information obtainment, lesion information obtainment, surface extraction, boundary definition, length measurement, area measurement, volume measurement, and/or annotation creation, from the raw image frames and/or ultrasonic images in various modes.

The AI model may be implemented using various artificial neural network models or deep neural network models. In addition, the AI model may be learned and created using various machine learning algorithms or deep learning algorithms. The AI model may be implemented using, for example, models such as a convolutional neural network (CNN), a recurrent neural network (RNN), a generative adversarial network (GAN), and a long short-term memory (LSTM).

As described above, the ultrasonic imaging system 100 may include at least one of the memories 111 and 150. For example, each of the probe 20 and the ultrasonic imaging apparatus 40 may include one or more memories. At least one of the memories 111 and 150 may include volatile memory (e.g., S-RAM and D-RAM) and/or non-volatile memory (e.g., ROM and EPROM).

The ultrasonic imaging system 100 may include at least one of the processors 118, 120, and 130. For example, each of the probe 20 and the ultrasonic imaging apparatus 40 may include one or more processors. At least one of the processors 118, 120, and 130 may include various processing circuits and/or multiple processors. At least one of the processors 118, 120, and 130 may be configured to perform various functions individually and/or collectively in a distributed manner.

The ultrasonic imaging system 100 may include a plurality of processors configured to individually and/or collectively perform various functions, or may include an integrated processor configured to be performable all of the various functions. For example, the processor 120 and the image processor 130 of the ultrasonic imaging apparatus 40 described above may be provided as separate processors or as one integrated processor.

At least one of the memories 111 and 150 may store various algorithms, instructions, commands, data and/or artificial intelligence models for controlling the probe 20 and processing ultrasonic raw data. At least one of the memories 111 and 150 may store an artificial intelligence model learned to perform an artificial intelligence-based function. At least one of the memories 111 and 150 may store additional information about the object including at least one of a subcutaneous fat thickness, a body mass index (BMI), gender, age, and an underlying disease, which is input through the input interfaces 109 and 170.

At least one of the processors 118, 120, and 130 may include one or more of a central processing unit (CPU), a graphics processing unit (GPU), an accelerated processing unit (APU), a many integrated core (MIC), a digital signal processor (DSP), a neural processing unit (NPU), a hardware accelerator, and a machine learning accelerator.

At least one of the processors 118, 120, and 130 may control the probe 20 and the ultrasonic imaging apparatus 40. At least one of the processors 118, 120, and 130 may control an operation of the ultrasonic imaging system 100 by executing various algorithms, instructions, commands, data, and/or artificial intelligence models stored in at least one of the memories 111 and 150.

At least one of the processors 118, 120, and 130 may perform operation control of the probe 20 and/or processing of ultrasonic raw data obtained by the probe 20. At least one of the processors 118, 120, and 130 may generate raw image frames including a variety of information and/or ultrasonic images in various modes using the ultrasonic raw data.

At least one of the processors 118, 120, and 130 may input raw image frames and/or ultrasonic images in various modes into the learned AI model and obtain results output from the AI model. For example, at least one of the processors 118, 120, and 130 may, using the AI model, perform quantitative data obtainment on diseases of various tissues, reference line definition, anatomical information obtainment, lesion information obtainment, surface extraction, boundary definition, length measurement, area measurement, volume measurement, and/or annotation creation, from the raw image frames and/or the ultrasonic images in various modes.

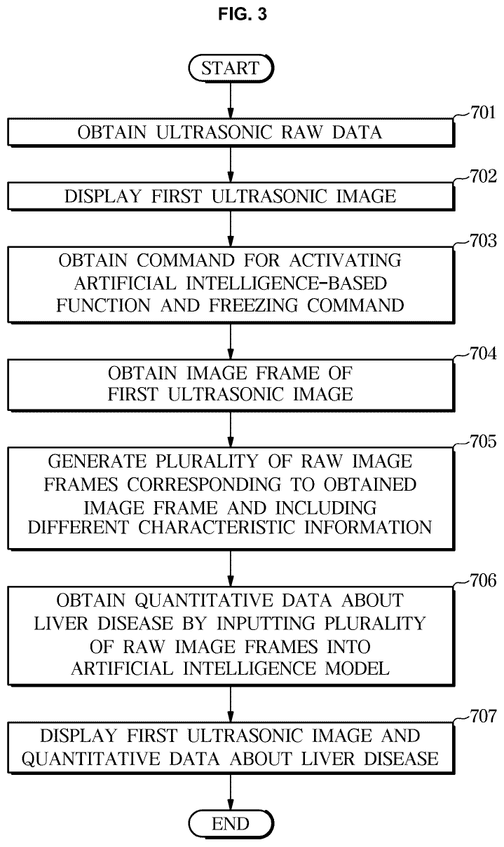

FIG. 3 is a flowchart for explaining a control method of an ultrasonic imaging system according to an embodiment.

Referring to FIG. 3, at least one of the processors 120 and 130 of the ultrasonic imaging system 100 may obtain ultrasonic raw data by processing an ultrasonic echo signal received through the probe 20 and reflected from the object (701). At least one of the processors 120 and 130 may generate ultrasonic raw data by converting the reception signals obtained from the probe 20 into analog to digital and summing up the digitally converted reception signals in consideration of the positions and focused points of the plurality of transducers.

At least one of the processors 120 and 130 may display a first ultrasonic image generated by processing the ultrasonic raw data on the display 140 (702). At least one of the processors 120 and 130 may generate the first ultrasonic image in various modes by processing the ultrasonic raw data. The first ultrasonic image may be a video including a plurality of image frames. The first ultrasonic image may be displayed on at least one of the main display 140a and the sub display 140b of the ultrasonic image apparatus 40.

For example, at least one of the processors 120 and 130 may generate an ultrasonic image in the amplitude mode (A-mode), the brightness mode (B-mode), the color Doppler mode, the Doppler mode (D-mode), the elastography mode (E-mode), the motion mode (M-mode), and/or the volume mode by processing the ultrasonic raw data. Depending on a mode for the ultrasonic image, the method of processing the ultrasonic raw data may vary.

In an embodiment, the first ultrasonic image may be a ‘B-mode image’. At least one of the processors 120 and 130 may perform the following processes to generate the B-mode image from the ultrasonic raw data (i.e., RF data):

1) envelope detection to extract amplitude information from the ultrasonic raw data (i.e., RF data); 2) logarithmic compression to compress a dynamic range of a signal by which the envelope detection has been performed; 3) time gain compensation to compensate for signal attenuation according to depth for a signal by which the logarithmic compression has been performed; 4) spatial interpolation to fill an empty space between scan lines for a signal by which the time gain compensation has been performed; 5) post-processing for noise removal, edge emphasis, and contrast enhancement for a signal by which the spatial interpolation has been performed; and 6) convert a signal by which the post-processing has been performed into an 8-bit black-and-white image.

At least one of the processors 120 and 130 may obtain the freezing command through the input interface 109 of the probe 20 or the input interface 170 of the ultrasonic imaging apparatus 40. At least one of the processors 120 and 130 may also obtain a command for activating the artificial intelligence-based function through the input interface 109 of the probe 20 or the input interface 170 of the ultrasonic imaging apparatus 40 (703).

For example, the processor 120 of the ultrasonic imaging apparatus 40 may obtain the command for activating the artificial intelligence-based function through the artificial intelligence-based function button 173 provided on the control panel 165 or displayed on the display 140. The user may input the command for activating the artificial intelligence-based function by pressing or touching the artificial intelligence-based function button 173. The processor 120 may perform processes for providing the quantitative data about the liver disease through the display 140 using the artificial intelligence model in response to obtaining the command for activating the artificial intelligence-based function.

At least one of the processors 120 and 130 of the ultrasonic imaging system 100 may obtain an image frame of the first ultrasonic image in response to obtaining the freezing command through the input interfaces 109 and 170 (704). For example, the processors 120 and 130 of the ultrasonic imaging apparatus 40 may obtain the freezing command through the freeze button 172 provided on the control panel 165 or displayed on the display 140. The user may input the freezing command by pressing or touching the freeze button 172. The processors 120 and 130 may pause the display of the first ultrasonic image in response to obtaining the freezing command, and display the image frame at the point in time when the freezing command is obtained through the display 140. The processors 120 and 130 may capture the image frame at the point in time when the freezing command is obtained. The processors 120 and 130 may also store the image frame at the point in time when the freezing command is obtained.

At least one of the processors 120 and 130 of the ultrasonic imaging system 100 may generate a plurality of raw image frames corresponding to the image frame of the first ultrasonic image obtained by processing the ultrasonic raw data and including different characteristic information (705).

The generation of the raw image frame may be performed through a different process from generation of the first ultrasonic image. The raw image frame may not be displayed through the display 140, and the process of generating the raw image frame may not include at least one of the time gain compensation, spatial interpolation, noise removal, post-processing, and image conversion.

For example, at least one of the processors 120 and 130 may generate a first raw image frame including attenuation information of the ultrasonic echo signal (RF signal) and a second raw image frame including scattering information of the ultrasonic echo signal, as the plurality of raw image frames.

In addition, at least one of the processors 120 and 130 may further generate at least one of a third raw image frame including in-phase component information and quadrature component information of the ultrasonic raw data and a fourth raw image frame including frequency spectrum information of the ultrasonic raw data, as the plurality of raw image frames. At least one of the processors 120 and 130 may generate the third raw image frame for independently visualizing an amplitude and phase information by decomposing the ultrasonic raw data into I (in-phase) and Q (quadrature) components. At least one of the processors 120 and 130 may generate the fourth raw image frame for observing characteristics of a specific frequency band by applying a Fourier transform to the ultrasonic raw data.

The plurality of raw image frames is not limited to those exemplified. The ultrasonic imaging system 100 may further generate other raw image frames including other information in addition to the exemplified raw image frames. Characteristic information included in the raw image frame may vary depending on user input. The user may select characteristic information of a raw image frame desired through the input interfaces 109 and 170.

At least one of the processors 120 and 130 of the ultrasonic imaging system 100 may input the plurality of raw image frames into the artificial intelligence model and obtain the quantitative data about the liver disease from the artificial intelligence model (706). For example, at least one of the processors 120 and 130 may input all of the raw image frames or part of the raw image frames into the artificial intelligence model. The quantitative data about the liver disease may include at least one of a fat fraction of a liver and severity of liver steatosis.

At least one of the processors 120 and 130 may identify a first region of interest in the image frame of the first ultrasonic image and determine a second region of interest corresponding to the first region of interest in each of the plurality of raw image frames. For example, at least one of the processors 120 and 130 may obtain first coordinate information of the first region of interest in the image frame of the first ultrasonic image and convert the first coordinate information into second coordinate information in each of the plurality of raw image frames. At least one of the processors 120 and 130 may determine the second region of interest based on the second coordinate information. At least one of the processors 120 and 130 may extract a partial image frame corresponding to the second region of interest in each of the plurality of raw image frames. At least one of the processors 120 and 130 may obtain the quantitative data about the liver disease in the first region of interest by inputting a plurality of the partial image frames corresponding to each of a plurality of the second regions of interest into the artificial intelligence model.

In addition, at least one of the processors 118, 120, and 130 may obtain additional information about the object including at least one of the subcutaneous fat thickness, the body mass index (BMI), the gender, the age, and the underlying disease from the memory 150 or the input interfaces 109 and 170. At least one of the processors 118, 120, and 130 may input the plurality of raw image frames and the additional information about the object into the artificial intelligence model in order to obtain the quantitative data about the liver disease.

At least one of the processors 118, 120, and 130 of the ultrasonic imaging system 100 may display the first ultrasonic image and the quantitative data about the liver disease together on the displays 112 and 140 (707). For example, at least one of the processors 118, 120, and 130 may display a second ultrasonic image including the quantitative data about the liver disease on the displays 112 and 140. At least one of the processors 118, 120, and 130 may display the quantitative data about the liver disease in at least one of a first region of the display 140 displaying the first ultrasonic image and the second ultrasonic image and a second region of the displays 112 and 140 divided from the first region. At least one of the processors 118, 120, and 130 may also display a heat map visualizing a distribution of the quantitative data about the liver disease on the displays 112 and 140.

As such, the disclosed ultrasonic imaging system 100 may generate a raw image including a variety of information by processing the ultrasonic raw data, and obtain the quantitative data about the liver disease by inputting the generated raw image into the artificial intelligence model. The ultrasonic imaging system 100 may also reduce a data processing amount of the artificial intelligence model by using the raw image generated by pre-processing the ultrasonic raw data as input to the artificial intelligence model. The ultrasonic imaging system 100 may also provide the user even with information that may not be checked in various existing ultrasonic imaging modes by using the ultrasonic raw data and the artificial intelligence model.

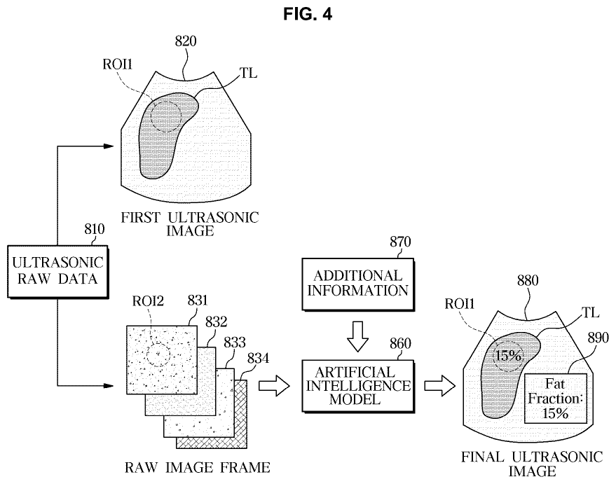

FIG. 4 is a diagram for explaining a process in which an artificial intelligence-based function of the ultrasonic imaging system according to an embodiment is performed.

Referring to FIG. 4, the ultrasonic imaging apparatus 40 constituting the ultrasonic imaging system 100 may control the probe 20 to transmit an ultrasonic signal to an object including the liver (TL). The ultrasonic imaging apparatus 40 may obtain ultrasonic raw data 810 by processing ultrasonic echo signals (i.e., RF signals) received through the probe 20 and reflected from the object. The ultrasonic raw data 810 may also be referred to as RF data. The processor 120 of the ultrasonic imaging apparatus 40 may generate the ultrasonic raw data 810 by converting the RF signals into analog to digital and summing up the digitally converted reception signals in consideration of the positions and focused points of the plurality of transducers.

The image processor 130 of the ultrasonic imaging apparatus 40 may generate a first ultrasonic image 820 in various modes by processing the ultrasonic raw data 810. The processor 120 of the ultrasonic imaging apparatus 40 may display a first region of interest ROI1 on the first ultrasonic image 820. For example, the image processor 130 may generate an ultrasonic image in the amplitude mode (A-mode), the brightness mode (B-mode), the color Doppler mode, the Doppler mode (D-mode), the elastography mode (E-mode), the motion mode (M-mode), and/or the volume mode by processing the ultrasonic raw data. The mode of the first ultrasonic image 820 may vary depending on user input obtained through the input interfaces 109 and 170. The first ultrasonic image 820 may be the ‘B-mode image’.

The processor 120 may set the first region of interest ROI1 based on the user input obtained through the input interfaces 109 and 170. The processor 120 may also set the first region of interest ROI1 using a learning model for detecting a region of interest. For example, the first ultrasonic image 820 may be input to the learning model, and the first region of interest ROI1 may be set in the first ultrasonic image 820 based on output of the learning model. The first region of interest ROI1 may be set in the entire region of the liver TL, or may be set in at least a partial region of the liver TL.

The processor 120 of the ultrasonic imaging apparatus 40 may obtain an image frame of the first ultrasonic image 820 in response to obtaining the freezing command through the input interfaces 109 and 170. The processors 120 and 130 may pause the display of the first ultrasonic image 820 in response to obtaining the freezing command, and display the image frame at the point in time when the freezing command is obtained through the display 140.

The processor 120 of the ultrasonic imaging apparatus 40 may obtain the command for activating the artificial intelligence-based function through the artificial intelligence-based function button 173 provided on the control panel 165 or displayed on the display 140. The processor 120 may perform the processes for providing the quantitative data about the liver disease through the display 140 using the artificial intelligence model in response to obtaining the command for activating the artificial intelligence-based function.

The image processor 130 of the ultrasonic imaging apparatus 40 may generate a plurality of raw image frames 831, 832, 833, and 834 corresponding to the image frame of the obtained first ultrasonic image 820 and including different characteristic information. The processor 120 may control the image processor 130 to generate the plurality of raw image frames 831, 832, 833, and 834.

For example, the image processor 130 may generate the first raw image frame 831 including the attenuation information of the ultrasonic echo signal (RF signal) and the second raw image frame 832 including the scattering information of the ultrasonic echo signal, as the plurality of raw image frames 831, 832, 833, and 834.

The first raw image frame 833 may correspond to a power spectrum density map (PSD map) for observing attenuation characteristics of the RF signal in a depth direction. The image processor 130 may Fourier transform the ultrasonic raw data 810 in the depth direction, calculate a power value according to a frequency and depth, and generate the PSD map for multiple frequency bands. Because the more fat a tissue (e.g., liver) has, the more ultrasonic energy is absorbed, power attenuation occurs more rapidly with depth. A different attenuation pattern may be detected for each frequency band of the RF signal.

The second raw image frame 832 may be obtained through a Nakagami Imaging process for analyzing scattering characteristics of the tissue. The image processor 130 may set a specific region (e.g., ROI) by obtaining envelope data of the ultrasonic raw data 810, and may generate an image including the scattering information by analyzing an intensity distribution of the RF signal in the specific region and calculating a Nakagami parameter. A fat content of the tissue (e.g., liver) may be estimated from a value of the Nakagami parameter.

In addition, the image processor 130 may further generate at least one of the third raw image frame 833 including in-phase component information and quadrature component information of the ultrasonic raw data 810 and the fourth raw image frame 834 including frequency spectrum information of the ultrasonic raw data 810. The image processor 130 may generate the third raw image frame 833 for independently visualizing an amplitude and phase information by decomposing the ultrasonic raw data into I (in-phase) and Q (quadrature) components. The image processor 130 may generate the fourth raw image frame 834 for observing characteristics of a specific frequency band by applying a Fourier transform to the ultrasonic raw data.

The generation of the raw image frames 831, 832, 833, and 834 may be performed through a different process from generation of the first ultrasonic image 820. The raw image frame may not be displayed through the display 140, and the process of generating the raw image frame may not include at least one of the time gain compensation, spatial interpolation, noise removal, post-processing, and image conversion.

The plurality of raw image frames is not limited to those exemplified. The ultrasonic imaging system 100 may further generate other raw image frames including other information in addition to the exemplified raw image frames.

The processor 120 of the ultrasonic imaging apparatus 40 may input the plurality of raw image frames 831, 832, 833, and 834 into an artificial intelligence model 860. The processor 120 may input all of the raw image frames 831, 832, 833, and 834 or part of the raw image frames 831, 832, 833, and 834 into the artificial intelligence model 860.

For example, the image processor 120 may identify the first region of interest ROI1 in the image frame of the first ultrasonic image 820 and determine a second region of interest ROI2 corresponding to the first region of interest ROI1 in each of the plurality of raw image frames 831, 832, 833, and 834. The processor 120 may obtain first coordinate information of the first region of interest ROI1 in the image frame of the first ultrasonic image 820 and convert the first coordinate information into second coordinate information in each of the plurality of raw image frames 831, 832, 833, and 834. The processor 120 may determine the second region of interest ROI2 in each of the plurality of raw image frames 831, 832, 833, and 834 based on the second coordinate information. The processor 120 may extract a partial image frame corresponding to the second region of interest ROI2 in each of the plurality of raw image frames 831, 832, 833, and 834. The processor 120 may input a plurality of the partial image frames corresponding to each of a plurality of the second regions of interest ROI2 into the artificial intelligence model 860.

The processor 120 may obtain the quantitative data about the liver disease in the first region of interest ROI1 of the first ultrasonic image 820 by inputting all of the raw image frames 831, 832, 833, and 834 or the plurality of the partial image frames corresponding to each of the plurality of second regions of interest ROI2 into the artificial intelligence model 860. For example, the quantitative data about the liver disease may include at least one of the fat fraction of the liver and the severity of the liver steatosis.

The plurality of raw image frames 831, 832, 833, and 834 may not be displayed through the display 140. The processor 120 may perform other image processing to generate the first ultrasonic image 820 and the plurality of raw image frames 831, 832, 833, and 834.

A plurality of the first regions of interest ROI1 may be set at different locations of the liver TL in the first ultrasonic image 820. In this case, several number of the second regions of interest ROI2 may be set at different locations in each of the plurality of raw image frames 831, 832, 833, and 834. In other words, as the locations and/or number of the first regions of interest ROI1 set in the first ultrasonic image 820 are changed, the locations and/or number of the second regions of interest ROI2 may be changed in each of the raw image frames 831, 832, 833, and 834.

In addition, the processor 120 of the ultrasonic imaging apparatus 40 may obtain additional information 870 about the object including at least one of the subcutaneous fat thickness, the body mass index (BMI), the gender, the age, and the underlying disease from the memory 150 or the input interface 109 and 170. The processor 120 may input the plurality of raw image frames 831, 832, 833, and 834 and the additional information 870 about the object into the artificial intelligence model 860 in order to obtain the quantitative data about the liver disease. As the additional information 870 about the object is input into the artificial intelligence model 860, the quantitative data about the liver disease may be obtained more accurately.

The processor 120 of the ultrasonic imaging apparatus 40 may control the display 140 to display a final ultrasonic image 880 including the quantitative data about the liver disease. The final ultrasonic image 880 may be a still image corresponding to the image frame of the first ultrasonic image 820 obtained in response to the freezing command. The final ultrasonic image 880 may be referred to as the second ultrasonic image.

The quantitative data about the liver disease may be displayed at various locations within the final ultrasonic image 880. For example, the quantitative data about the liver disease may be displayed within the first region of interest ROI1. The quantitative data about the liver disease may also be displayed in an information display region 890, which is a different region from the first region of interest ROI1 within the final ultrasonic image 880.

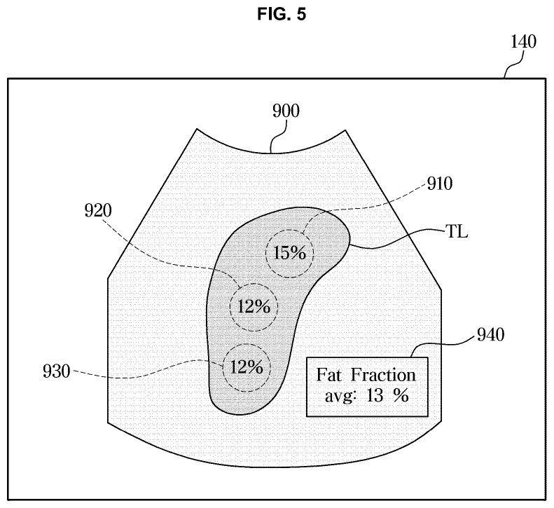

FIG. 5 illustrates an example of an aspect in which an ultrasonic image and quantitative data about the liver disease are displayed through a display of the ultrasonic imaging apparatus.

Referring to FIG. 5, the processor 120 of the ultrasonic imaging apparatus 40 may set a plurality of the first regions of interest at different locations of the liver TL in the first ultrasonic image 820. For example, the processor 120 may set each of an upper region 910, middle region 920, and lower region 930 of the liver TL as the first region of interest. The processor 120 may automatically set a plurality of regions of interest at various locations in the liver TL using the learning model for detecting regions of interest according to user input or without user input.

The processor 120 may control the display 140 to display a final ultrasonic image 900 including the quantitative data about diseases in the upper region 910, middle region 920, and lower region 930 of the liver TL. For example, the ultrasonic imaging apparatus 40 may display the final ultrasonic image 900 including a fat fraction for each of the upper region 910, middle region 920, and lower region 930 of the liver TL. The final ultrasonic image 900 may be a still image corresponding to the image frame of the first ultrasonic image 820 obtained in response to the freezing command. The final ultrasonic image 900 may be referred to as the second ultrasonic image.

For example, the processor 120 may determine a plurality of the second regions of interest corresponding to the upper region 910, middle region 920, and lower region 930 of the liver TL in each of the plurality of raw image frames 831, 832, 833, and 834. For example, the processor 120 may set the second region of interest at a location corresponding to each of the upper region 910, middle region 920, and lower region 930 of the liver TL in each of the first raw image frame 831 and the second raw image frame 832.

The processor 120 may extract a partial image frame corresponding to each of the upper region 910, middle region 920, and lower region 930 of the liver TL in each of the plurality of raw image frames 831, 832, 833, and 834. The processor 120 may input a plurality of the partial image frames to the artificial intelligence model 860.

The artificial intelligence model 860 may output quantitative data about disease in each of the upper region 910, middle region 920, and lower region 930 of the liver TL. The processor 120 of the ultrasonic imaging apparatus 40 may control the display 140 to display the quantitative data about disease in each of the upper region 910, middle region 920, and lower region 930 of the liver TL.

For example, the fat fractions for the upper region 910, middle region 920, and lower region 930 of the liver TL within the final ultrasonic image 900 may be individually displayed in the upper region 910, middle region 920, and lower region 930, respectively. In addition, an average values for the fat fractions for the upper region 910, middle region 920, and lower region 930 of the liver TL may be displayed in an information display region 940 of the final ultrasonic image 900.

In addition, the ultrasonic imaging apparatus 40 may display the severity of the liver steatosis in each of the upper region 910, middle region 920 and lower region 930 of the liver TL.

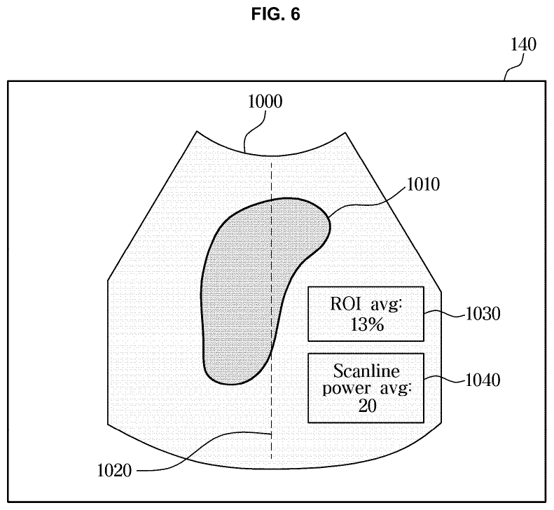

FIG. 6 illustrates an example of an aspect in which an ultrasonic image and quantitative data about a liver disease are displayed through the display of the ultrasonic imaging apparatus.

Referring to FIG. 6, the processor 120 of the ultrasonic imaging apparatus 40 may set an entire region 1010 of the liver TL in the first ultrasonic image 820 as the first region of interest. The processor 120 may automatically set the entire region 1010 of the liver TL as the first region of interest according to user input or without user input.

The processor 120 may determine the second region of interest corresponding to the entire region 1010 of the liver TL in each of the plurality of raw image frames 831, 832, 833, and 834. The processor 120 may extract a partial image frame corresponding to the entire region 1010 of the liver TL in each of the plurality of raw image frames 831, 832, 833, and 834. The processor 120 may input a plurality of the partial image frames into the artificial intelligence model 860. The artificial intelligence model 860 may output quantitative data about a disease in the entire region 1010 of the liver TL.

The processor 120 may control the display 140 to display a final ultrasonic image 1000 including quantitative data about the disease in the entire region 1010 of the liver TL. The final ultrasonic image 1000 may be a still image corresponding to the image frame of the first ultrasonic image 820 obtained in response to the freezing command. The final ultrasonic image 1000 may be referred to as the second ultrasonic image. For example, the ultrasonic imaging apparatus 40 may display an average value of a fat fraction for the entire region 1010 of the liver TL and an average value of RF signal power in the depth direction along a scan line 1020.

Within the final ultrasonic image 1000, the average value of the fat fraction for the entire region 1010 of the liver TL may be displayed in first information display region 1030, and the average value of the RF signal power may be displayed in second information display region 1040.

In addition, the ultrasonic imaging apparatus 40 may display the severity of the liver steatosis in the entire region 1010 of the liver TL.

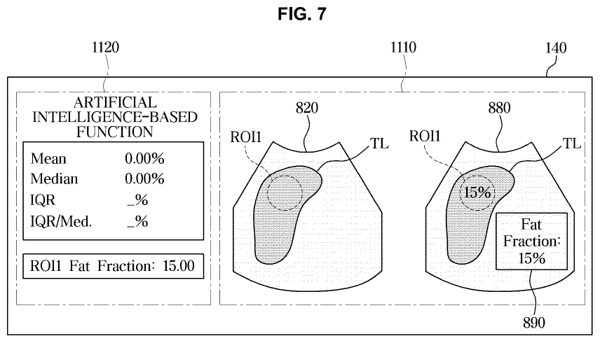

FIG. 7 illustrates an example of an aspect in which an ultrasonic image and quantitative data about a liver disease are displayed through the display of the ultrasonic imaging apparatus.

Referring to FIG. 7, the processor 120 of the ultrasonic imaging apparatus 40 may control the display 140 to display the first ultrasonic image 820 and the final ultrasonic image 880 including the quantitative data regarding the liver disease together. The processor 120 may simultaneously display the first ultrasonic image 820 and the final ultrasonic image 880 in a first region 1110 of the display 140. The final ultrasonic image 880 may be referred to as the second ultrasonic image.

In addition, the processor 120 may display the quantitative data about the liver disease in a second region 1120 of the display 140 divided from the first region 1110. The ultrasonic imaging apparatus 40 may store the final ultrasonic images 880 and the quantitative data about the liver disease. Even when the freezing command for displaying the first ultrasonic image 820 is released, the previously obtained quantitative data about the liver disease may continue to be displayed in the second region 1120 of the display 140.

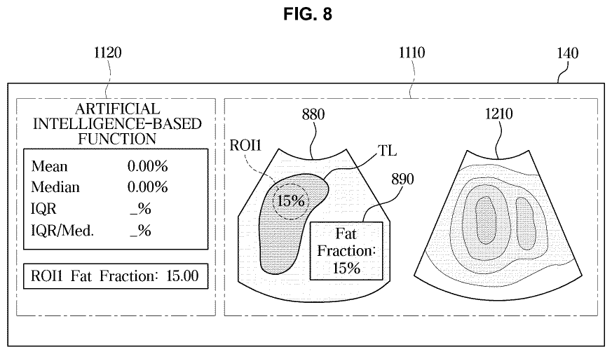

FIG. 8 illustrates an example of an aspect in which an ultrasonic image and quantitative data about a liver disease are displayed through the display of the ultrasonic imaging apparatus.

Referring to FIG. 8, the processor 120 of the ultrasonic imaging apparatus 40 may control the display 140 to display the final ultrasonic image 880 including the quantitative data about the liver disease and a heat map 1210 visualizing the distribution of the quantitative data about the liver disease. For example, the processor 120 may simultaneously display the final ultrasonic image 880 and the heat map 1210 in the first region 1110 of the display 140. The heat map 1210 may be displayed to overlap the first ultrasonic image 820 or the final ultrasonic image 880.

Aspects of a screen provided through the display 140 of the ultrasonic imaging apparatus 40 are not limited to those exemplified. The ultrasonic image and the quantitative data about liver disease may be presented on various screens depending on designs.

As is apparent from the above, the disclosed ultrasonic imaging system and control method thereof can obtain quantitative data about a liver disease from ultrasonic raw data using an artificial intelligence model, and display the obtained data together with an ultrasonic image.

The disclosed ultrasonic imaging system and control method thereof can generate a raw image including a variety of information by processing the ultrasonic raw data, and obtain the quantitative data about the liver disease by inputting the generated raw image into the artificial intelligence model.

The disclosed ultrasonic imaging system and control method thereof can, by using the raw image generated by pre-processing the ultrasonic raw data as input to the artificial intelligence model, reduce an amount of data processing of the artificial intelligence model and provide a user even with information that cannot be checked in various existing ultrasonic imaging modes.

In addition, the disclosed ultrasonic imaging system and control method thereof can minimize user intervention for obtaining data using the artificial intelligence model and provide data together with the ultrasonic image familiar to the user. Through this, consistency of obtained data can be ensured and user convenience can be improved.

The disclosed embodiments may be implemented in the form of a recording medium storing instructions executable by a computer. The instructions may be stored in the form of program code, and when executed by a processor, a program module may be created to perform the operations of the disclosed embodiments. The recording medium may be implemented as a computer-readable recording medium.

The computer-readable recording medium includes any type of recording medium in which instructions readable by the computer are stored. For example, the recording medium may include a read only memory (ROM), a random access memory (RAM), a magnetic tape, a magnetic disk, a flash memory, an optical data storage device, and the like.

In addition, the device-readable recording medium may be provided in the form of a non-transitory storage medium. Herein, the ‘non-transitory storage medium’ simply means that it is a tangible device and does not contain signals (e.g. electromagnetic waves), and this term does not distinguish between a case in which data is semi-permanently stored in a storage medium and a case in which data is stored temporarily. For example, the ‘non-transitory storage medium’ may include a buffer in which data is temporarily stored.

According to an embodiment, the methods according to various embodiments disclosed in this document may be included and provided in a computer program product. The computer program product is a commodity and may be traded between sellers and buyers. The computer program product may be distributed in the form of a machine-readable recording medium (e.g., compact disc read only memory (CD-ROM)), or may be distributed (e.g., downloaded or uploaded) online, through an application store (e.g., Play Store™) or directly between two user devices (e.g., smartphones). In the case of online distribution, at least a portion of the computer program product (e.g., a downloadable application) may be at least temporarily stored or created temporarily in the machine-readable recording medium, such as the memory of a manufacturer server, an application store server, and a relay server.

The embodiments disclosed with reference to the accompanying drawings have been described above. It will be understood by those skilled in the art that various changes in form and details may be made therein without departing from the spirit and scope of the disclosure as defined by the appended claims. The disclosed embodiments are illustrative and should not be construed as limiting.

What is claimed is:

1. An ultrasonic imaging system comprising:
a probe configured to transmit an ultrasonic signal to an object comprising a liver and receive an ultrasonic echo signal reflected from the object;
a display configured to display an ultrasonic image;
an input interface configured to obtain user input;
memory configured to store an artificial intelligence model; and
at least one processor,
wherein the at least one processor is configured to
obtain ultrasonic raw data by processing the ultrasonic echo signal,
display a first ultrasonic image generated by processing the ultrasonic raw data on the display,
obtain an image frame of the first ultrasonic image in response to obtaining a freezing command through the input interface,
generate a plurality of raw image frames corresponding to the obtained image frame and comprising different characteristic information by processing the ultrasonic raw data,
obtain quantitative data about a liver disease from the artificial intelligence model by inputting the plurality of raw image frames into the artificial intelligence model, and
display a second ultrasonic image comprising the quantitative data about the liver disease on the display.

2. The ultrasonic imaging system according to claim 1, wherein
the at least one processor is configured to
generate a B-mode image as the first ultrasonic image, and
generate a first raw image frame comprising attenuation information of the ultrasonic echo signal and a second raw image frame comprising scattering information of the ultrasonic echo signal, as the plurality of raw image frames.

3. The ultrasonic imaging system according to claim 2, wherein
the at least one processor is configured to
further generate at least one of a third raw image frame comprising in-phase component information and quadrature component information of the ultrasonic raw data and a fourth raw image frame comprising frequency spectrum information of the ultrasonic raw data, as the plurality of raw image frames.

4. The ultrasonic imaging system according to claim 1, wherein
the at least one processor is configured to
identify a first region of interest in the image frame of the first ultrasonic image,
determine a second region of interest corresponding to the first region of interest in each of the plurality of raw image frames,
extract a partial image frame corresponding to the second region of interest in each of the plurality of raw image frames, and
obtain the quantitative data about the liver disease in the first region of interest by inputting a plurality of the partial image frames corresponding to each of a plurality of the second regions of interest into the artificial intelligence model.

5. The ultrasonic imaging system according to claim 4, wherein
the at least one processor is configured to
obtain first coordinate information of the first region of interest in the image frame of the first ultrasonic image,
convert the first coordinate information into second coordinate information in each of the plurality of raw image frames, and
determine the second region of interest based on the second coordinate information.

6. The ultrasonic imaging system according to claim 1, wherein
the at least one processor is configured to
obtain at least one of a fat fraction of the liver and severity of liver steatosis as the quantitative data about the liver disease.

7. The ultrasonic imaging system according to claim 1, wherein
the at least one processor is configured to
obtain additional information about the object comprising at least one of a subcutaneous fat thickness, a body mass index (BMI), gender, age, and an underlying disease through the input interface, and
input the plurality of raw image frames and the additional information about the object into the artificial intelligence model.

8. The ultrasonic imaging system according to claim 1, wherein
the at least one processor is configured to
display the quantitative data about the liver disease in at least one of a first region of the display displaying the first ultrasonic image and the second ultrasonic image and a second region of the display divided from the first region.

9. The ultrasonic imaging system according to claim 1, wherein
the at least one processor is configured to
further display a heat map visualizing a distribution of the quantitative data about the liver disease on the display.

10. The ultrasonic imaging system according to claim 1, wherein
the at least one processor is configured to
display the second ultrasonic image on the display based on obtaining a command for activating an artificial intelligence-based function through the input interface.

11. A control method of an ultrasonic imaging system, which comprises a probe, an input interface, a display, and at least one processor, comprising a control method executed by the at least one processor,
wherein the control method comprises:
controlling the probe to transmit an ultrasonic signal to an object comprising a liver and receive an ultrasonic echo signal reflected from the object;
obtaining ultrasonic raw data by processing the ultrasonic echo signal;
displaying a first ultrasonic image generated by processing the ultrasonic raw data on the display;
obtaining an image frame of the first ultrasonic image in response to obtaining a freezing command through the input interface;
generating a plurality of raw image frames corresponding to the obtained image frame and comprising different characteristic information by processing the ultrasonic raw data;
obtaining quantitative data about a liver disease from the artificial intelligence model by inputting the plurality of raw image frames into the artificial intelligence model; and
displaying a second ultrasonic image comprising the quantitative data about the liver disease on the display.

12. The control method according to claim 11, wherein
the first ultrasonic image corresponds to a B-mode image, and
the generating of the plurality of raw image frames comprises
generating a first raw image frame comprising attenuation information of the ultrasonic echo signal and a second raw image frame comprising scattering information of the ultrasonic echo signal.

13. The control method according to claim 12, wherein
the generating of the plurality of raw image frames further comprises
generating at least one of a third raw image frame comprising in-phase component information and quadrature component information of the ultrasonic raw data and a fourth raw image frame comprising frequency spectrum information of the ultrasonic raw data, as the plurality of raw image frames.

14. The control method according to claim 11, wherein
the obtaining of the quantitative data about the liver disease comprises:
identifying a first region of interest in the image frame of the first ultrasonic image;
determining a second region of interest corresponding to the first region of interest in each of the plurality of raw image frames;
extracting a partial image frame corresponding to the second region of interest in each of the plurality of raw image frames; and
obtaining the quantitative data about the liver disease in the first region of interest by inputting a plurality of the partial image frames corresponding to each of a plurality of the second regions of interest into the artificial intelligence model.

15. The control method according to claim 14, wherein
the determining of the second region of interest comprises:
obtaining first coordinate information of the first region of interest in the image frame of the first ultrasonic image;
converting the first coordinate information into second coordinate information in each of the plurality of raw image frames; and
determining the second region of interest based on the second coordinate information.

16. The control method according to claim 11, wherein
the quantitative data about the liver disease comprises at least one of a fat fraction of the liver and severity of liver steatosis.

17. The control method according to claim 11, further comprising:
obtaining additional information about the object comprising at least one of a subcutaneous fat thickness, a body mass index (BMI), gender, age, and an underlying disease through the input interface; and
inputting the plurality of raw image frames and the additional information about the object into the artificial intelligence model.

18. The control method according to claim 11, wherein
the displaying of the second ultrasonic image comprises
displaying the quantitative data about the liver disease in at least one of a first region of the display displaying the first ultrasonic image and the second ultrasonic image and a second region of the display divided from the first region.

19. The control method according to claim 11, further comprising
displaying a heat map visualizing a distribution of the quantitative data about the liver disease on the display.

20. The control method according to claim 11, wherein
the displaying of the second ultrasonic image is performed based on obtaining a command for activating an artificial intelligence-based function through the input interface.
